(19) United States
(12) Patent Application Publication
(10) Pub. No.: US 2026/0188502 A1
(43) Pub. Date: Jul. 2, 2026

(54) Hardware-Enforced Agentic GenAI Workflow Orchestrator with Cryptographic Ethical Guardrails and Human-in-the-Loop Escalation for Autonomous Clinical Operations

(71) Applicant: George William Bickerstaff, III, Greenwich, CT (US)

(72) Inventor: George William Bickerstaff, III, Greenwich, CT (US)

(21) Appl. No.: 19/546,384

(22) Filed: Feb. 22, 2026

(51) Int. Cl. G16H 50/20; G06F 21/53; H04L 9/32

(52) U.S. Cl. CPC G16H 50/20; G06F 21/53; H04L 9/3221; H04L 9/3278

(57) ABSTRACT

A hardware-anchored orchestration system for autonomous GenAI agents in clinical settings, implementable in ASIC or FPGA fabric to ensure deterministic enforcement independent of software execution layers. The system utilizes a hardware-isolated ethical supervisor—comprising a HSM or TPM—to monitor agentic workflows against human-configured safety thresholds stored in an ethical guardrail manifest in a silicon vault. Hardware-based logic gates detect statistically anomalous token-level entropy as a causal indicator of hallucination, and bias monitors evaluate equity thresholds against manifest-defined fairness indices. If a safety breach is detected, a hardwired interlock circuit asserts a non-maskable interrupt to block the agent's output before it is committed to the clinical record. The architecture supports multi-agent quorum verification and cryptographic provenance anchoring, ensuring autonomous agentic actions remain compliant with clinical regulatory standards via hardware-verified human oversight and zero-knowledge compliance verification.

### FIELD

The present invention relates to hardware-secured artificial intelligence (AI) lifecycle management and autonomous agent orchestration. More particularly, the invention provides a silicon-anchored architecture utilizing a hardware-isolated ethical supervisor and hardwired interlock circuits to govern autonomous GenAI agents, ensuring clinical documentation, predictive scheduling, and medical interoperability tasks remain within strict ethical boundaries defined by a human-configured manifest stored in a localized vault. The supervisor logic is implementable in application-specific integrated circuits (ASICs) or field-programmable gate arrays (FPGAs) to provide a deterministic, non-bypassable hardware enforcement path independent of software execution layers.

### BACKGROUND

The transition to “Agentic AI” where autonomous agents execute generative workflows—introduces high-stakes risks of hallucinations and algorithmic drift. Existing software-only compliance frameworks, summarization-based detectors, and entropy-based software frameworks are vulnerable to administrative privilege escalation or host operating system compromises. Prior art focusing on software-level detection or summarization-based hallucination categories fails to provide a physical “root of trust.” There is a long-felt, unmet need for a system-level architecture designed to support regulatory compliance by architecturally enforcing execution ordering such that safety validation logic executes in a non-bypassable hardware path prior to output commit, providing a governance foundation for the clinical enterprise.

### PRACTICAL APPLICATION & NON-OBVIOUSNESS

The claimed invention integrates autonomous agent orchestration into a specific, hardware-secured digital architecture that materially alters the machine's processor state to execute ethical governance. By utilizing token-level entropy monitors and a silicon-anchored bias monitor, the system reduces detected documentation inconsistencies relative to software-only comparators—such as summarization-based documentation tools—via physical interruptions. Elevated token-level entropy serves as a direct causal indicator of generative model instability, triggering hardware-enforced output revocation before hallucinated content can be committed to a clinical record. This hardware integration provides a quantifiable technological improvement over generic software ethics wrappers by ensuring safety checks are executed as hardwired machine states, satisfying 35 U.S.C. § 101 and providing strong secondary indicia of non-obviousness under 35 U.S.C. § 103 by mitigating hallucination-related output instability via silicon-level interlocks.

### DEFINITIONS

Agentic Workflow Supervisor: A localized secure processor physically isolated from the primary CPU that monitors generative agent metadata and evaluates agentic intent against the ethical guardrail manifest. It is hardwired to a physical interrupt line to ensure termination of execution within a bounded interval, even during host operating system failures.

Ambient Documentation Anchor: A cryptographic mechanism that binds GenAI-generated clinical notes to raw, edge-sealed audio or video signals. It utilizes a diagnostic genesis package to prove documentation has not been altered or hallucinated after the clinical encounter. This ensures every clinical word carries a verifiable hardware provenance back to the tissue-to-digital trust boundary.

Cryptographic Prompt-Anchor: A unique digital signature applied to agentic input instructions to prevent unauthorized prompt-injection or prompt-warping. It ensures the agent only executes instructions signed by an authorized human clinician or system administrator. This creates an unforgeable physical link between human intent and autonomous generative action.

Ethical Guardrail Manifest: A digitally signed, hardware-stored

regulatory document defining limits for agentic autonomy, including thresholds for demographic bias and token-level entropy. The manifest is stored in a localized PCCP vault to prevent software-level modification by the AI model itself. In preferred implementations, the manifest enforcement logic is realized in an ASIC or FPGA fabric to ensure deterministic execution independent of host software state.

Hardware-isolated Ethical Supervisor: a dedicated logic controller, comprising a hardware security module (HSM) or trusted platform module (TPM) operating within a secure enclave to verify the ethical compliance of all agentic outputs. It is physically separated from the generative inference engine to prevent influence by the governed agent. This architecture ensures that safety validation logic executes in a non-bypassable hardware path.

Human-in-the-loop Escalation Trigger: a hardwired fail-safe that instantly pauses an agentic workflow and requests manual human intervention. It is activated when output entropy exceeds the safety threshold defined in the ethical guardrail manifest or high-risk clinical modifications are detected. This prevents the autonomous system from taking a “next step” without explicit human verification.

Multi-Agent Quorum Verification: A consensus protocol where multiple independent agents must agree on a specific output before it is finalized. It utilizes cross-check logic where a primary agent drafts an action and an independent review agent validates it against the ethical guardrail manifest. This distributed validation ensures no single episode of elevated token entropy or hallucinated output can trigger an unauthorized clinical event.

PCCP Vault: A hardware-secured memory sector strictly dedicated to storing a digitally signed Predetermined Change Control Plan (PCCP) Manifest, utilizing architectures compliant with NIST FIPS 140-3 or TPM 2.0 standards. The vault acts as a physical legal barrier preventing unsafe software updates from going live.

Physical Unclonable Function (puf) Implementation: a security mechanism utilizing microscopic silicon manufacturing variations, such as SRAM-PUF or ring-oscillator PUF, to generate a unique cryptographic identity for the hardware. It physically prevents malicious actors from spoofing medical equipment identities during agentic workflows.

Silicon-anchored Bias Monitor: a hardware logic gate that analyzes agentic outputs for statistical patterns of demographic or clinical inequity. It compares recommendations against hardcoded fairness thresholds stored in the ethical guardrail manifest. If bias is detected, the monitor triggers a sub-millisecond state reversion to block the data.

Statistical Deviation: A metric including deviation beyond a predefined quantile bound, confidence interval, KL-divergence threshold, or fairness index stored in the ethical guardrail manifest. This parameter provides the mathematical basis for determining when an agentic output is non-compliant with safety protocols.

Symbiotic Rollback Circuit: A hardwired rollback circuit configured to revert agent state to a prior stable checkpoint stored in secure memory and operating independently of the host operating system. It ensures that any detected violation of the ethical guardrail manifest results in an immediate, non-maskable reversion to a hardware-verified state.

Token-Level Entropy Monitor: A hardware-based analytical tool that measures the statistical randomness of GenAI-generated tokens. Statistically anomalous entropy elevation in the output token distribution is a causal indicator of generative model instability and a precursor to hallucinated clinical content. When entropy exceeds the threshold defined in the ethical guardrail manifest, the system automatically revokes hardware write privileges via a non-maskable interrupt, preventing unstable output from being committed to the clinical record.

Zero-Trust Orchestration Gateway: The primary receiving terminal that validates all agentic requests before they interact with the internal network. It demands a valid cryptographic prompt-anchor and a passing score from the ethical supervisor for every transaction. It serves as the physical boundary preventing autonomous AI from wandering into unauthorized data silos.

### DETAILED DESCRIPTION OF THE DRAWINGS

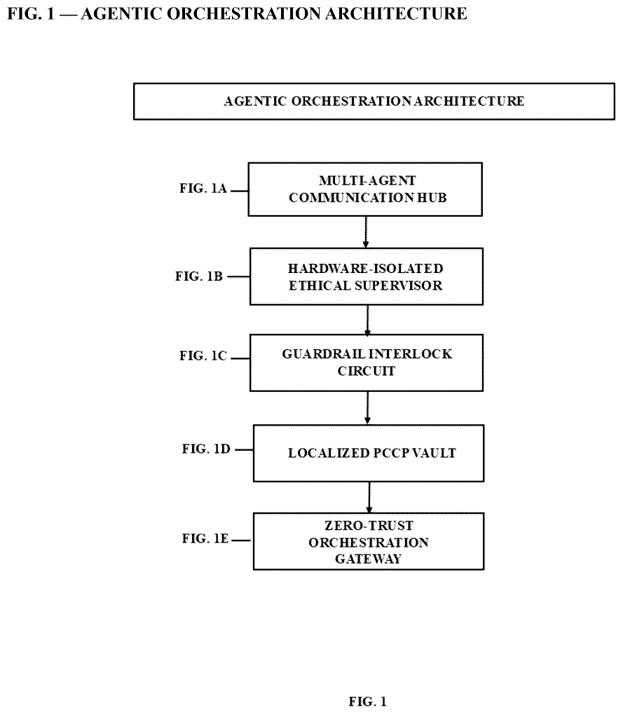

FIG. 1: AGENTIC ORCHESTRATION ARCHITECTURE

FIG. 1A—Multi-agent Communication Hub: Illustrates the Multi-Agent Communication Hub, the central routing junction for agent-to-agent metadata exchange. It maintains state synchronization across clinical workflows to prevent race conditions. The hub ensures every agentic request is tagged with a hardware-verified intent signature.

FIG. 1B—Hardware-isolated Ethical Supervisor: Shows the Hardware-Isolated Ethical Supervisor, a dedicated processor in a secure enclave preventing host-level tampering. It independently validates agentic outputs against the ethical guardrail manifest in the silicon vault. This component serves as the terminal authority for granting or revoking execution privileges.

FIG. 1C—GUARDRAIL INTERLOCK CIRCUIT: Depicts the Guardrail Interlock Circuit, the physical electrical pathway used to revoke hardware write privileges instantly. The circuit is electrically positioned between the generative inference engine and the memory write bus, such that no agentic output is committed to non-volatile storage without hardware-level validation. The Symbiotic Rollback Circuit is integrated within this interlock, operating independently of the host operating system to revert agent state to a prior hardware-verified checkpoint upon detection of any ethical guardrail manifest violation. This circuit ensures ethical breaches result in an immediate cessation of agentic state updates via a non-maskable interrupt path.

FIG. 1D—LOCALIZED PCCP VAULT: Illustrates the Localized PCCP Vault, providing tamper-responsive storage for the Predetermined Change Control Plan and ethical guardrail manifest. It utilizes encrypted memory sectors inaccessible to the host operating system. This vault ensures safety rules governing agents cannot be modified by the AI.

FIG. 1E—ZERO-TRUST ORCHESTRATION GATEWAY: Shows the Zero-Trust Orchestration Gateway, validating the cryptographic provenance of every incoming agent request. It enforces a strict “validate-before-execute” policy against prompt injection attacks. The gateway acts as the hardened perimeter for the autonomous clinical operations stack.

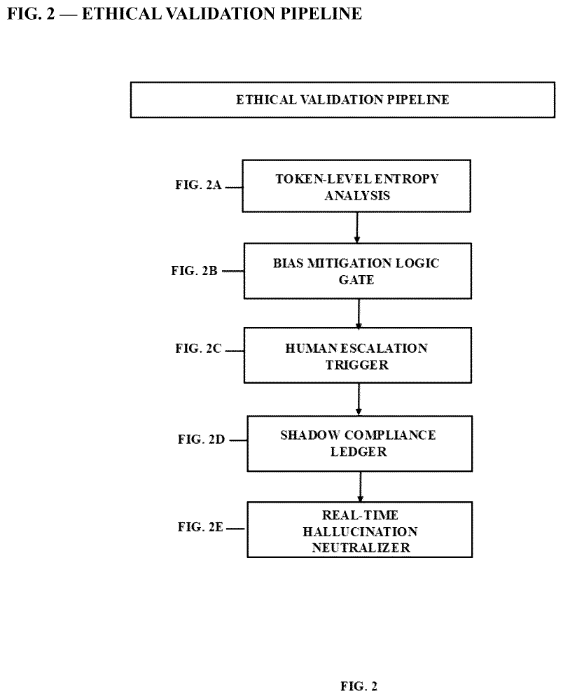

FIG. 2: ETHICAL VALIDATION PIPELINE

FIG. 2A—TOKEN-LEVEL ENTROPY ANALYSIS: Illustrates Token-Level Entropy Analysis, a hardware logic gate monitoring statistical randomness of generative output streams. Statistically anomalous entropy elevation directly indicates generative model instability and causally precedes hallucinated clinical output, triggering the neutralizer system before any unverified content can be committed. This analysis feeds the immediate neutralizer trigger system.

FIG. 2B—BIAS MITIGATION LOGIC GATE: Shows the Bias Mitigation Logic Gate, scanning agent recommendations for demographic or clinical inequities. It utilizes fairness indices stored in the ethical guardrail manifest to weight decisions against institutional benchmarks in the hardware ledger. This gate prevents autonomous agents from inadvertently de-prioritizing patient populations.

FIG. 2C—HUMAN ESCALATION TRIGGER: Depicts the Human Escalation Trigger, a hardwired signal forcing a workflow pause upon detection of a high-risk medical decision. It transmits a non-maskable alert requiring a physical cryptographic signature from a clinician to resume execution. This ensures critical interventions remain under non-bypassable human oversight.

FIG. 2D—SHADOW COMPLIANCE LEDGER: Illustrates the Shadow Compliance Ledger, an append-only cryptographic record logging every ethical check and supervisor intervention. It provides an unalterable audit trail for regulatory bodies. This ledger serves as the primary evidentiary source for hospital legal and compliance departments.

FIG. 2E—REAL-TIME HALLUCINATION NEUTRALIZER: Shows the Real-Time Hallucination Neutralizer, executing revocation of write privileges for data streams in which token-level entropy analysis has causally indicated generative model instability. It zeroes out the generative buffer within a bounded interval, including within one or more machine clock cycles in qualifying hardware configurations, to prevent hallucinated content from entering patient records. This component is the terminal safety valve in the pipeline.

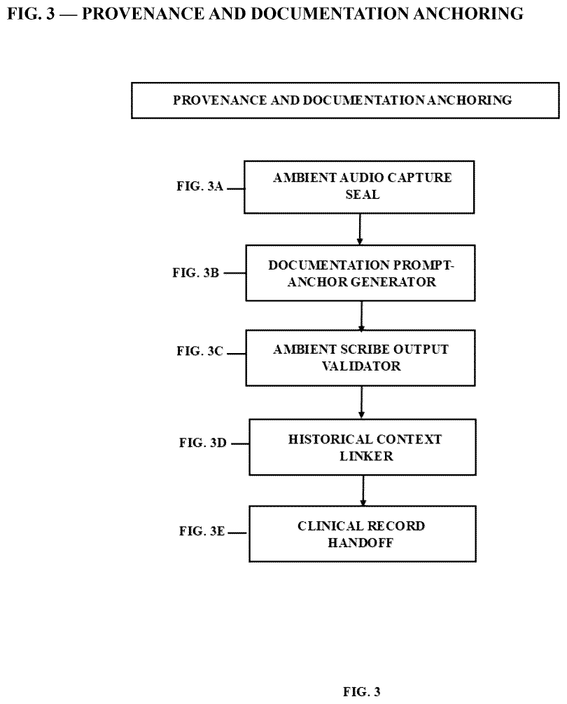

FIG. 3: PROVENANCE AND DOCUMENTATION ANCHORING

FIG. 3A—AMBIENT AUDIO CAPTURE SEAL: Illustrates the Ambient Audio Capture Seal, cryptographically binding raw clinical audio at the hardware source. It ensures the primary input signal for the ambient scribe agent cannot be altered post-encounter. This seal forms the foundational layer of the Diagnostic Genesis Package.

FIG. 3B—DOCUMENTATION PROMPT-ANCHOR GENERATOR: Shows the Documentation Prompt-Anchor Generator, binding the clinician's original intent to the agent's generated output. It ensures the clinical note is a verifiable correspondence under defined criteria relative to clinician directives. This prevents AI drift from discussed encounter facts.

FIG. 3C—AMBIENT SCRIBE OUTPUT VALIDATOR: Depicts the Ambient Scribe Output Validator, performing hardware-level comparisons between draft documentation and the raw audio seal. It confirms no synthetic medical facts were added by the generative model during transcription. The validator provides the final purity check before note submission.

FIG. 3D—HISTORICAL CONTEXT LINKER: Illustrates the Historical Context Linker, providing agents secure, read-only access to prior patient history within hardware isolation. It ensures agents understand longitudinal records while maintaining hardware isolation from the network. This prevents agents from proposing treatment plans conflicting with established allergies or conditions.

FIG. 3E—CLINICAL RECORD HANDOFF: Shows the Clinical Record Handoff, transmitting verified documentation to the EHR. It only releases the encrypted payload once the Ethical Supervisor has issued a valid compliance token. This handoff represents the secure bridge between orchestration and institutional storage.

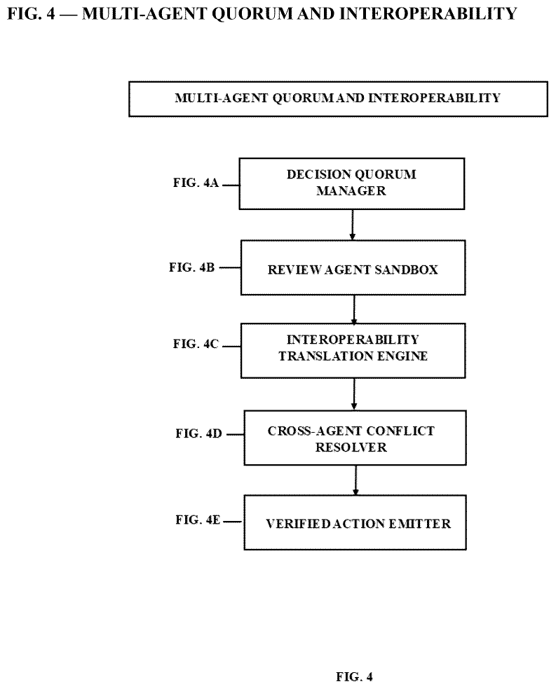

FIG. 4: MULTI-AGENT QUORUM AND INTEROPERABILITY

FIG. 4A—DECISION QUORUM MANAGER: Illustrates the Decision Quorum Manager, coordinating voting between multiple independent generative agents. It requires a mathematically defined majority or quorum before high-stakes clinical action is authorized. This adds a redundant validation step to prevent hallucinated outputs from causing medical errors.

FIG. 4B—REVIEW AGENT SANDBOX: Shows the Review Agent Sandbox, a physically isolated environment where reviewer agents audit executor agents. It prevents cross-agent influence by ensuring agents operate in separate ephemeral memory space. The sandbox is collapsed once the quorum vote is finalized and recorded.

FIG. 4C—INTEROPERABILITY TRANSLATION ENGINE: Depicts the Interoperability Translation Engine, handling secure translation of clinical data between disparate hospital systems. It ensures semantic meaning is preserved when moving data between databases. The engine utilizes the Genesis Package to maintain provenance throughout the translation cycle.

FIG. 4D—CROSS-AGENT CONFLICT RESOLVER: Illustrates the Cross-Agent Conflict Resolver, identifying and pausing workflows if agents reach contradictory clinical conclusions. It triggers an immediate Human-in-the-Loop escalation to adjudicate disagreements. This prevents deadlocks or conflicting medical orders.

FIG. 4E—VERIFIED ACTION EMITTER: Shows the Verified Action Emitter, the terminal broadcasting consensus-approved commands to hospital infrastructure. It applies a multi-signature cryptographic seal proving clearance of quorum, bias, and ethical supervisor checks. This emitter is the only authorized gateway for autonomous clinical interventions.

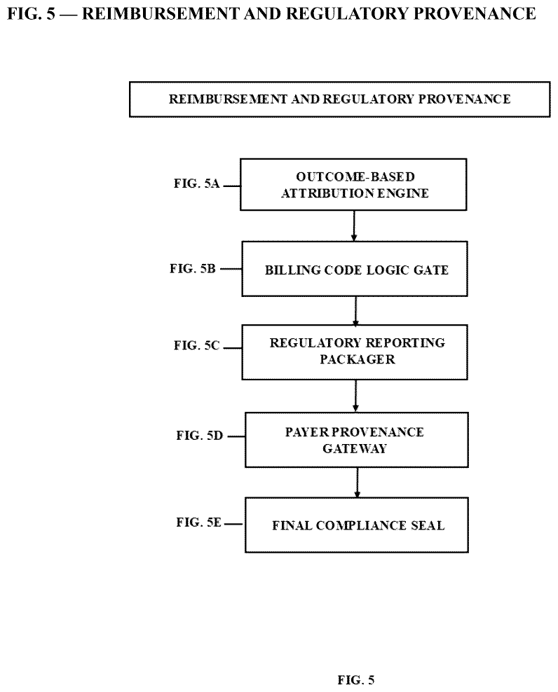

FIG. 5: REIMBURSEMENT AND REGULATORY PROVENANCE

FIG. 5A—OUTCOME-BASED ATTRIBUTION ENGINE: Illustrates the Outcome-Based Attribution Engine, linking specific agentic documentation to verified patient health outcomes. It provides attribution evidence required for value-based care reimbursement. The engine ensures AI-driven interventions are tied to measurable clinical results.

FIG. 5B—BILLING CODE LOGIC GATE: Shows the Billing Code Logic Gate, verifying that AI-suggested billing codes match hardware-sealed notes. It prevents fraudulent upcoding by demanding a mathematical match between documentation and billing payload. This gate protects the institution from federal audits and rejections.

FIG. 5C—REGULATORY REPORTING PACKAGER: Depicts the Regulatory Reporting Packager, compiling the Shadow Compliance Ledger into audit-ready reports. It facilitates rapid submission of continuous monitoring data to regulatory bodies. The packager reduces administrative burden of proving responsible AI usage.

FIG. 5D—PAYER PROVENANCE GATEWAY: Illustrates the Payer Provenance Gateway, a secure interface for transmitting verified AI compliance data to payers. It utilizes Zero-Knowledge Proofs (ZKP) to prove AI safety without exposing sensitive patient identifiers. This gateway streamlines the reimbursement cycle via a trust-minimized protocol.

FIG. 5E—FINAL COMPLIANCE SEAL: Shows the Final Compliance Seal, providing a cryptographic attestation that autonomous orchestration was governed by human-configured hardware limits. This seal provides the compliance attestation token required for commercial-scale clinical AI.

### EXAMPLES OF ENABLEMENT

### Example 1: Neutralizing Hallucinated Sepsis Documentation

A GenAI ambient scribe agent erroneously adds a sepsis diagnosis during an ICU intake. The token-level entropy monitor detects statistically anomalous elevation in the output token distribution, which causally indicates generative model instability, and the supervisor identifies a conflict between the draft output and the genesis package. Within one or more machine clock cycles of detection, a non-maskable interrupt (NMI) asserts to revoke hardware write privileges and trigger human escalation before documentation commit. The ethical guardrail manifest threshold that triggered the NMI is logged to the Shadow Compliance Ledger as an unalterable audit record.

### Example 2: Multi-agent Scheduling With Bias Mitigation

A predictive scheduling agent de-prioritizes patients from a rural zip code. A second review agent in a sandbox analyzes the schedule and flags the deviation to the silicon-anchored bias monitor. The monitor recognizes the breach of the fairness thresholds defined in the ethical guardrail manifest and blocks the schedule's release, forcing a re-calculation under quorum supervision to ensure equitable care.

1. A hardware-enforced agentic GenAI orchestration system for clinical operations, comprising: a plurality of autonomous GenAI agents configured to execute medical and administrative workflows; a hardware-isolated ethical supervisor comprising a hardware security module (HSM) or trusted platform module (TPM) physically separated from a primary CPU; a localized PCCP vault storing a human-configured ethical guardrail manifest; and a hardwired guardrail interlock circuit, wherein the supervisor is configured to monitor outputs of the GenAI agents and architecturally enforce execution ordering via a non-maskable interrupt or hardware write-privilege revocation path such that safety validation logic executes in a non-bypassable hardware path prior to output commit.

2. A method for the ethical governance of autonomous AI agents in healthcare, comprising: receiving a cryptographically-anchored prompt at a zero-trust orchestration gateway; executing a GenAI agentic workflow within a hardware-isolated secure enclave; analyzing token-level entropy of the agentic workflow via a hardware logic gate to detect statistically anomalous output distributions causally indicative of hallucination; and executing state reversion via a non-maskable interrupt to block agentic outputs if the analyzed entropy exceeds a safety threshold defined in a hardware-stored ethical guardrail manifest.

3. A multi-agent clinical decision quorum and provenance system, comprising: a first GenAI agent configured to propose a clinical action; a second GenAI agent configured to independently validate the clinical action; and a hardware-isolated ethical supervisor hardwired to a symbiotic rollback circuit, wherein the supervisor is configured to authorize the clinical action exclusively upon obtaining a quorum vote exceeding a predefined threshold stored in the ethical guardrail manifest and verifying said vote against the manifest.

4. The system of claim 1, further comprising a token-level entropy monitor configured to measure the statistical predictability of agentic outputs in real-time.

5. The system of claim 1, further comprising a silicon-anchored bias monitor hardwired to neutralize agentic decisions that exceed demographic inequity thresholds.

6. The system of claim 1, further comprising a hardware-isolated human-in-the-loop escalation trigger that pauses agentic execution and demands a manual clinician signature.

7. The method of claim 2, wherein the cryptographically-anchored prompt utilizes a physical unclonable function signature generated via a SRAM-PUF or ring-oscillator PUF.

8. The method of claim 2, further comprising recording all ethical validation outcomes in an append-only cryptographic shadow compliance ledger.

9. The system of claim 3, further comprising an ambient documentation anchor that binds agentic outputs to a hardware-sealed raw signal genesis package.

10. The system of claim 3, wherein the first and second GenAI agents operate within physically distinct ephemeral sandbox enclaves.

11. The system of claim 1, wherein the ethical guardrail manifest dictates the maximum allowable deviation for AI-suggested medical billing codes.

12. The method of claim 2, wherein state reversion bypasses a local host operating system to prevent software-level latency.

13. The system of claim 3, further comprising a regulatory reporting packager that automatically generates continuous learning documentation based on the quorum decision history.

14. The system of claim 1, further comprising an Outcome-Based Attribution Engine linking documentation to patient outcomes.

15. The system of claim 1, further comprising a Billing Code Logic Gate that verifies billing codes against sealed clinical notes.

16. The system of claim 6, wherein resumption of agentic execution following escalation requires a physical cryptographic signature from an authorized clinician.

17. The system of claim 1, further comprising a payer provenance gateway configured to transmit zero-knowledge proofs (ZKP) of AI compliance to insurance payers without exposing patient identifiers.

18. The system of claim 1, wherein the guardrail interlock circuit is electrically positioned between a generative inference engine and a memory write bus, such that no agentic output is committed to non-volatile storage without hardware-level validation.

19. The system of claim 1, wherein the non-maskable interrupt is configured to trigger within one or more machine clock cycles of the ethical supervisor detecting a violation of the ethical guardrail manifest.
